(19) United States
(12) Patent Application Publication
(10) Pub. No.: US 2010/0159010 A1
(43) Pub. Date: Jun. 24, 2010

(54) Active Agent Formulations, Methods of Making, and Methods of Use

(75) Inventors: Maher S. Kudsi, Cherry Hill, NJ (US); Rakeshkumar K. Lad, Marlton, NJ (US); Hengsheng Feng, Cherry Hill, NJ (US)

Correspondence Address: CANTOR COLBURN, LLP, 20 Church Street, 22nd Floor, Hartford, CT, 06103, US

(73) Assignee: MUTUAL PHARMACEUTICAL COMPANY, INC., Philadelphia, PA (US)

(21) Appl. No.: 12/644,202

(22) Filed: Dec. 22, 2009

Related U.S. Application Data
(60) Provisional application No. 61/140,700, filed on Dec. 24, 2008.

(51) Int. Cl. A61K 9/00; A61K 47/32; A61K 9/14; A61P 1/00

(52) U.S. Cl. 424/484; 514/772.4; 424/501

(57) ABSTRACT

An active agent composition includes particles containing a water-insoluble active agent or an active agent with a significant food effect; and a ternary amine polymer having both hydrophobic (meth)acrylate units and acid-soluble (meth)acrylate units, and an acidifying agent; wherein upon ingestion by a human subject, the acidifying agent facilitates the dissolution of the ternary amine polymer in the gastrointestinal tract.

### CROSS-REFERENCE TO RELATED APPLICATIONS

This application claims the benefit of U.S. Provisional Application Ser. No. 61,140,700, filed Dec. 24, 2008, which is incorporated by reference herein in its entirety.

### BACKGROUND

Bioavailability means the extent and/or rate at which an active agent is absorbed into a living system, or is made available at the site of physiological activity. Many factors can affect bioavailability including the dosage form and various properties of the active agent and/or dosage form, e.g., dissolution rate of the active agent. Poor bioavailability is a significant problem encountered in the development of pharmaceutical compositions, particularly those containing an active agent that is poorly soluble in water. Poorly water-soluble active agents can be eliminated from the gastrointestinal tract before being absorbed into the circulation. It is known that the rate of dissolution of a particulate active agent can increase with increasing surface area, i.e., decreasing particle size.

The present invention addresses the need for improved compositions comprising insoluble active agents.

### SUMMARY

In one embodiment, an active agent composition comprises particles comprising a water-insoluble active agent or an active agent with a significant food effect; and a ternary amine polymer having both hydrophobic (meth)acrylate units and acid-soluble (meth)acrylate units, and an acidifying agent; wherein upon ingestion by a human subject, the acidifying agent facilitates the dissolution of the ternary amine polymer in the gastrointestinal tract.

In another embodiment, a method of producing a solid dosage form, comprises

(a) selecting a ternary amine polymer;

(b) selecting an acidifying agent, which has a rate of dissolution suitable to facilitate dissolution of the ternary amine polymer under aqueous acidic conditions;

(c) producing particles comprising a water-insoluble active agent or an active agent with a significant food effect, and the ternary amine polymer; and

(d) combining the particles with the acidifying agent to produce the dosage form, wherein upon ingestion of the dosage form by a human subject, the acidifying agent facilitates the dissolution of the ternary amine polymer in the gastrointestinal tract.

These and other embodiments, advantages and features of the present invention are illustrated by the Detailed Description and Examples that follow.

### DETAILED DESCRIPTION

Disclosed herein are compositions comprising particles containing a water-insoluble active agent or an active agent with a significant food effect; and a ternary amine polymer having both hydrophobic (meth)acrylate units and acid-soluble (meth)acrylate units, wherein the composition further comprises an acidifying agent; wherein upon ingestion by a human subject, the acidifying agent facilitates the dissolution of the ternary amine polymer in the gastrointestinal tract. In one embodiment, the acidifying agent is in the form of a matrix in which the particles are dispersed. In one embodiment, the dosage form is not a taste-masked dosage form. Advantageously, in certain embodiments the compositions are bioequivalent when dosed under fasted and non-fasted conditions.

It has been unexpectedly found by the inventors herein that the addition of an acidifying agent such as organic acid to an active agent particulate composition comprising a ternary amine polymer can facilitate the release of active agent from the particles. Without being held to theory, it is believed that for ternary amine polymers, e.g., Eudragit® E-100 or Eudragit® EPO, the inclusion of an acidifying agent and optionally a salt in the dosage form will extend the polymer dissolution solubility range to higher pH by affecting the “micro-environment” pH at the polymer surface. Improving the dissolution of the ternary amine polymer can improve the release of the active agent from the dosage form within the gastrointestinal tract. The chemical structure of Eudragit® E-100 is given below.

### 2 Chemical Structure

EUDRAGIT® E 100 is a cationic copolymer based on dimethylaminoethyl methacrylate, butyl methacrylate, and methyl methacrylate.

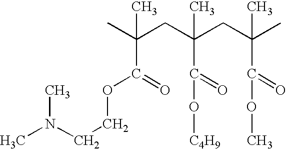

The average molecular weight is approx. 150,000.

The acidifying agent and optional salt are added to the particles during tabletting or capsule formation, during formation of the particles, during deposition of active agent, e.g., nanoparticles or microparticles, onto an inert core particle, or a combination thereof. When the acidifying agent and optional salt are added during tablet or capsule formation, they may be in the form of a matrix.

An acidifying agent is an agent that lowers pH. In one embodiment, the acidifying agent comprises an organic acid such as citric acid, maleic acid, fumaric acid, succinic acid, and combinations comprising one or more of the foregoing acids. In another embodiment, the acid comprises fumaric acid, succinic acid, or a combination thereof. The ratio of the acidifying agent to the ternary amine polymer is about 0.1 to about 10.0, specifically about 0.3 to about 2.

In another embodiment, the composition optionally comprises an ioinic strength modifier such as a salt. Exemplary salts include, for example, sodium salts such as sodium chloride. The optional salt is added at a concentration of about 0.1 N to about 1.0 N.

Disclosed herein are compositions comprising ternary amine polymers having both hydrophobic (meth)acrylate units and acid-soluble (meth)acrylate units. As used herein, (meth)acrylate encompasses both acrylates and methacrylates. Hydrophobic (meth)acrylate units are derived from (meth)acrylate monomers having a water solubility of less than or equal to 2 g per 100 g of water, measured at 25° C., specifically less than or equal to 1.5 g, more specifically less than or equal to 1.0 g. Acid-soluble (meth)acrylate units are derived from monomers containing basic groups, for example amines, and impart solubility and/or swellability to the polymer when in aqueous media having a pH of less than 5.5, specifically less than 5.0, more specifically less than 4.5, and even more specifically less than 4.0. In one embodiment the pH sensitive copolymer solubilizes or swells at a pH of about 3, as found in the stomach, but remains insoluble or deswelled at pH's greater than 4. Other types of units can be present in the polymer, provided that such units do not substantially adversely impact the sequestering activity of the polymer.

Exemplary (meth)acrylate monomers having a water solubility of 2 g or less per 100 g of water, measured at 25° C. include the C₁₋₁₈ hydrocarbyl esters of (meth)acrylic acid. “Hydrocarbyl” as used herein includes alkyl, cycloalkyl, alkylaryl, arylalkyl, and aryl groups that are unsubstituted or substituted with up to two heteroatoms, including halogen (fluorine, chlorine, bromine and iodine), nitrogen, oxygen, and sulfur. It is to be understood that any substituent (e.g., a hydroxy group) that increases the solubility of the monomer to above 2 g/100 g of water is not within the scope of the present compounds. Specific exemplary C₁₋₁₂ hydrocarbyl esters include methyl(meth)acrylate, ethyl(meth)acrylate, n-propyl(meth)acrylate, 2-propyl(meth)acrylate, cyclohexyl(meth)acrylate, dodecyl(meth)acrylate, 2-ethylhexyl(meth)acrylate, octyl(meth)acrylate, t-butyl(meth)acrylate n-butyl(meth)acrylate, phenyl(meth)acrylate, butyl(meth)acrylate, methyl methacrylate, benzyl(meth)acrylate, phenyl(meth)acrylate, and propyl methacrylate. Specific monomers are t-butyl(meth)acrylate, methyl methacrylate, and n-butyl(meth)acrylate.

In one embodiment, a combination of hydrophobic (meth)acrylate monomers is used. A specific combination comprises a hydrophobic (meth)acrylate monomer having a solubility of 1 to 2 g/100 g of water at 20° C., and a hydrophobic (meth)acrylate monomer having a solubility of less than 1 g/100 g of water at 20° C. An exemplary combination of hydrophobic (meth)acrylate monomers is a combination of methyl(meth)acrylate and butyl(meth)acrylate. The relative molar ratio of the hydrophobic (meth)acrylate having a solubility of 1 to 2 g/100 g of water at 20° C. to hydrophobic (meth)acrylate having a solubility of less than 1 g/100 g of water at 20° C., can vary widely depending on the active agent, the formulation solvent, availability, and like considerations, and can readily be determined by one of ordinary skill in the art without undue experimentation. In general, the molar ratio of the hydrophobic (meth)acrylate having a solubility of 1 to 2 g/100 g of water at 20° C. to hydrophobic (meth)acrylate having a solubility of less than 1 g/100 g of water at 20° C. is 95:5 to 5:95, specifically 80:20 to 20:80, more specifically 70:30 to 30:70.

Exemplary (meth)acrylate monomers containing basic groups are copolymerizable with the hydrophobic (meth)acrylate monomers, and have a functional group having a pKb of less than 20, specifically less than 10, more specifically less than 5. Nitrogen-containing functional groups are preferred. Tertiary amines are particularly useful, wherein the amine is connected to the (meth)acrylate via one of the amine substituents, and each of the substituents is the same or different. Exemplary substituents include C₁₋₁₂ hydrocarbyl groups, specifically unsubstituted C₁₋₁₂ hydrocarbyl groups, and even more specifically unsubstituted C₁₋₁₂ alkyl or cycloalkyl groups.

Exemplary (meth)acrylate monomers containing basic groups include 2-dimethylamino methyl(meth)acrylate, 2-dimethylamino ethyl(meth)acrylate, 2-diethylamino ethyl(meth)acrylate, 2-piperidinyl ethyl(meth)acrylate, and 2-(di-tert-butylamino)ethyl(meth)acrylate, specifically 2-dimethylamino ethyl methacrylate and 2-diethylamino ethyl acrylate.

The relative molar ratios of the hydrophobic (meth)acrylate and (meth)acrylate containing a basic group can vary widely depending on the active agent, the formulation solvent, availability, and like considerations, and can readily be determined by one of ordinary skill in the art without undue experimentation. In general, the molar ratio of the hydrophobic (meth)acrylate and (meth)acrylate containing a basic group is 95:5 to 5:95, specifically 80:20 to 20:80, more specifically 70:30 to 50:50. The copolymer can have a molecular weight of 10,000 to 800,000, specifically 50,000 to 500,000.

A specific ternary amine polymer is a butyl methacrylate-(2-dimethylaminoethyl methacrylate)-methyl methacrylate copolymer (1:2:1) available in granular form under the trade name EUDRAGIT® E-100. This copolymer has a mean molecular weight of 150,000, a viscosity of 3-12 mPas at 20° C., a refractive index of N²⁰_{D}: 1.380-1.385 and a relative density of d²⁰₄: 0.810-0.820. The same polymer is available in powder form under the trade name EUDRAGIT® E PO. In one embodiment, the particle sequestrant consists essentially of a butyl methacrylate-(2-dimethylaminoethyl methacrylate)-methyl methacrylate copolymer (1:2:1), for example the copolymer having a mean molecular weight of 150,000, a viscosity of 3-12 mPas at 20° C., a refractive index of N²⁰_{D}: 1.380-1.385 and a relative density of d²⁰₄: 0.810-0.820. In another embodiment, the particle sequestrant consists of butyl methacrylate-(2-dimethylaminoethyl methacrylate)-methyl methacrylate copolymer (1:2:1), for example the copolymer having a mean molecular weight of 150,000, a viscosity of 3-12 mPas at 20° C., a refractive index of N²⁰_{D}: 1.380-1.385 and a relative density of d²⁰₄: 0.810-0.820.

The active agent and the ternary amine polymer are in the form of particles such as microparticles or nanoparticles. In one embodiment, the active agent and the ternary amine polymer, optionally in the form of nanoparticles or microparticles, are disposed on an inert core.

In one embodiment, the active agent and the ternary amine polymer are disposed on an inert core, for example, by spraying.

In one embodiment, particles include nanoparticles, or microparticles, optionally disposed on an inert core. As used herein, nanoparticles generally have an average particle size of less than about 2000 nm, while microparticles have an average particle size of about 2000 nm to about 200 micrometers, specifically about 2000 nm to about 50 micrometers.

Microparticles can be formed by employing a size reduction process that employs the input of mechanical energy such as micronization, homogenization, microfluidization, milling, such as media milling, precipitation such as from a liquefied gas, ball milling and the like. In these size reduction processes, a composition including the active agent, the ternary amine polymer, and optionally additional excipients and the acidifying agent is subjected to the size reduction process.

In one embodiment, the active agent and ternary amine polymer are co-micronized to form microparticles. In another embodiment, the active agent is micronized and then mixed with the ternary amine polymer, and optionally with the organic acid.

In one embodiment, the oral compositions contain active agent nanoparticles that have an average particle size of less than about 2000 nm (i.e., 2 microns), less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, or less than 400 nm, as measured by light-scattering methods, microscopy, or other appropriate methods. As used throughout this specification, “particle size” refers to the largest diameter (i.e., dimension) of the particle.

In one embodiment, the oral compositions contain active agent nanoparticles have an effective average particle size of less than about 2000 nm (i.e., 2 microns), less than about 1900 nm, less than about 1800 nm, less than about 1700 nm, less than about 1600 nm, less than about 1500 nm, less than about 1400 nm, less than about 1300 nm, less than about 1200 nm, less than about 1100 nm, less than about 1000 nm, less than about 900 nm, less than about 800 nm, less than about 700 nm, less than about 600 nm, less than about 500 nm, or less than 400 nm, as measured by light-scattering methods, microscopy, or other appropriate methods. By “an effective average particle size of less than about 2000 nm” it is meant that at least 50% of the active agent particles, have a particle size of less than the average, by weight, i.e., less than about 2000 nm, 1900 nm, 1800 nm, etc., when measured by the above-noted techniques. Preferably, at least about 70%, about 90%, or about 95% of the particles have a particle size of less than the effective average, i.e., less than about 2000 nm, 1900 nm, 1800 nm, 1700 nm, etc. As is understood in the art, the value for D₅₀ of a nanoparticulate active agent is the particle size below which 50% of the particles fall, by weight. Similarly, D₉₀ is the particle size below which 90% of the fibrate particles fall, by weight. In certain embodiments, average diameter is used interchangeably with average particle size.

Nanoparticulate active agents can further have a narrow particle size distribution. In particular, less than 25%, less than 15%, less than 10%, or less than 5% (by weight) of the particles have a particle size greater than 4 micrometers. In another embodiment, less than 25%, less than 15%, less than 10%, or less than 5% (by weight) of the particles have a particle size greater than 3 micrometers. In still another embodiment, less than 25%, less than 15%, less than 10%, or less than 5% (by weight) of the particles have a particle size greater than 2 micrometers. In another embodiment, less than 50%, less than 35%, less than 20%, or less than 10% (by weight) of the particles have a particle size greater than 1 micrometer. In another embodiment, less than 50%, less than 35%, less than 20%, or less than 10% (by weight) of the particles have a particle size greater than 0.5 micrometers.

In one embodiment, in order to obtain bioequivalency and/or redispersibility, the active agent composition comprises active agent nanoparticles as described above and a compound that sequesters the nanoparticles during at least a portion of the processing to form the compositions. The particle sequestrant provides, among other advantages, improved bioavailability of the poorly-water soluble active agent. Without being bound by theory, it is hypothesized that during formulation, the particle sequestrant isolates the nanoparticulate active agents from adjacent nanoparticles. Agglomeration and/or crystal growth of the particles during formulation is accordingly inhibited, so that nanoparticles (rather than larger particles) are provided to the body upon dissolution (or other type of delivery) of the dosage form. It is also possible that the particle sequestrant inhibits agglomeration and/or crystal growth of the poorly water-soluble nanoparticulate active agents during or immediately after dissolution or other delivery in the body.

Effective particle sequestrants include ternary amine polymers having both hydrophobic (meth)acrylate units and acid-soluble (meth)acrylate units as described above. The particle sequestrant and the nanoparticulate active agent can be formulated using a variety of methods. In one embodiment, the particle sequestrant and the bioactive agent are combined and processed using standard techniques for tablet, capsule, suspension, or liquid formulation. The relative ratio of active agent and particle sequestrant will vary depending on the particular active agent and particle sequestrant used, the size of the nanoparticles, the other components in the formulation, and like considerations. Generally the weight ratio of active agent to particle sequestrant is 99:1 to 50:50, specifically 95:5 more specifically 90:10.

In a variation of this embodiment, the nanoparticles contain no added surfactants. In another embodiment, the formulation comprises no added surfactant. As used herein, a surfactant is limited to amphipathic compounds (as opposed to polymers) that contain both a hydrophobic region and a hydrophilic region. Surfactants can be anionic, cationic, zwitterionic, or nonionic. Specific surfactants that are excluded from the scope of the composition in this embodiment are sodium lauryl sulfate, sodium dioctyl sulfosuccinate, and phospholipids (a class of lipids formed from a fatty acid, a phosphate group, a nitrogen-containing alcohol and a backbone such as a glycerol backbone or a sphingosine backbone).

In one embodiment, in one method of manufacture, the active agent particles are reduced in size in the presence of at least one particle sequestrant. Alternatively, the active agent particles are contacted with one or more particle sequestrants after attrition. Other compounds, such as a diluent, can be added to the active agent or active agent/particle particle sequestrant composition during the size reduction process. Dispersions can be manufactured continuously or in a batch mode. A Dyno-Mill, or other suitable media mill can be used for the milling. The mill can be equipped with a temperature controlling unit to maintain the process temperature inside the milling chamber. The temperature of the suspension container can also be controlled.

In one specific embodiment, the pH-sensitive copolymer is dissolved in an aqueous solution, for example a buffered aqueous solution having a pH that is suitable to dissolve the pH-sensitive copolymer. Optionally, an organic solvent such as a C₁₋₃ alcohol is added to the solution as a wetting agent or to help dissolve the polymer. The alcohol is added in an amount effective to act as a wetting agent, e.g., 1-50% by volume of the combination of alcohol and water.

The water insoluble active agent is separately suspended in water, a mixture of 1-50 volume percent of a C₁₋₃ alcohol in water, or in a portion of the aqueous solution comprising the pH-sensitive copolymer.

In another embodiment, an active agent nanoparticle comprises a surface stabilizer adsorbed onto the surface of the active agent nanoparticles as described in U.S. Pat. No. 7,276,249, incorporated herein by reference. Surface stabilizers include polymers, low molecular weight oligomers, natural products, and surfactants. Surface stabilizers include nonionic, anionic, cationic, ionic, and zwitterionic surfactants. U.S. Pat. No. 7,276,249 contains a list of suitable surface stabilizers. The ternary amine polymer can be used in addition to a surface stabilizer to form nanoparticles, added to nanoparticles when they are disposed onto an inert core, or used in a matrix comprising the particles.

In one embodiment, active agent microparticles or nanoparticles are coated onto an inert particle, optionally in the presence of a coating material, to form a granulate. Exemplary coating materials for the granulate include, for example, a surfactant, a water-soluble polymer, a water-insoluble polymer, or a combination comprising one or more of the foregoing coating materials. Exemplary surfactants include sodium lauryl sulfate. Exemplary water-soluble polymers include hydroxyethylcellulose, hydroxypropylcellulose, hydroxyethylmethylcellulose, hydroxypropylmethylcellulose, carboxymethylcellulose, sodium carboxymethylcellulose, polyethylene glycol, and combinations comprising one or more of the foregoing water soluble polymers. Exemplary water insoluble polymers include, for example, an acrylic polymer, an acrylic copolymer, such as a methacrylic acid-ethyl acrylate copolymer, ethyl cellulose, or a combination comprising one or more of the foregoing water insoluble polymers.

Exemplary inert particles are also hydrophilic, dissolving readily in the body, and include, for example, sugars such as lactose, mannitol, dextrose and sorbitol; microcrystalline cellulose; calcium phosphate; lactose; and combinations comprising one or more of the foregoing inert particles. In one embodiment, the inert particles have an average diameter of 5 to 500 μm. As used herein, “calcium phosphate” includes a variety of materials that calcium ions (Ca²⁺) together with orthophosphates (PO43−), metaphosphates, or pyrophosphates (P₂O₇⁴⁻) and optionally hydrogen, halogen ions, or hydroxide ions, for example tricalcium phosphate, dicalcium phosphate dihydrate, and dicalcium phosphate, anhydrous, available under the trade name A-Tab® from Innophos, Cranbery, N.J.

In one embodiment, an active agent nanoparticle or microparticle suspension is dispersed onto the surface of an inert core particle, for example, by spraying in a fluid bed processor.

In another specific embodiment, the particulate active agent compositions can be made by a process comprising forming an aqueous solution of the pH-sensitive copolymers having both hydrophobic (meth)acrylate units and acid-soluble (meth)acrylate units, e.g., EUDRAGIT® E-100 or EUDRAGIT® E PO; forming a suspension of active agent in the aqueous solution; mixing and milling the suspension to form an active agent nanoparticulate or microparticulate suspension; and spraying the active agent nanoparticulate suspension over a powder bed comprising the inert cores to form granules having a suspension of the EUDRAGIT® polymer and active agent dispersed on the surface of the inert cores. In one embodiment, the active agent suspension comprises active agent particles with a particle size of 200-700 nm, in particular an average particle size of 200-700 nm, and even more particularly an effective average particle size of 200-700 nm. The active agent particles further have a D₉₀ of not more than 1.5 micrometers. The particle size can be measured using a Malvern Mastersizer at a proper analysis mode. When a wet analysis mode is chosen, a dispersant is used.

In one embodiment, an active agent nanoparticle suspension comprises an aqueous particle sequestrant solution having dispersed therein active agent nanoparticles. In one embodiment, the suspension is free of any added solubilizing and/or stabilizing agents other than the particle sequestrant. In another embodiment, an active agent nanoparticle suspension consists essentially of an aqueous particle sequestrant solution having dispersed therein active agent nanoparticles. In another embodiment, an active agent nanoparticle suspension consists of an aqueous particle sequestrant solution having dispersed therein active agent nanoparticles. In one embodiment, an active agent nanoparticle suspension is stable for up to two weeks after a particle size is measured. By stable it is meant that the average or effective average particle size of the active agent nanoparticles changes by no more than 35% within 2 weeks of a first particle size measurement, specifically by no more than 15% within 2 weeks of a first particle size measurement. In another embodiment, the concentration of the particle sequestrant is 1% w/v to 25% w/v, specifically 3% w/v to 15% w/v and the concentration of active agent is 5% w/v to 45% w/v, specifically 10% to 25% w/v.

The active agent composition can be redispersible in a biorelevant media such that the average or effective average particle size of the redispersed active agent particles is less than about 2000 nm. Redispersion of the active agent particles to a substantially nanoparticulate particle size preserves the benefits afforded by formulating the active agent into a nanoparticulate particle size. This is because nanoparticulate active agent compositions typically benefit from the small particle size of the active agent; if the active agent does not redisperse into the small particle sizes upon administration, then “clumps” or agglomerated active agent particles are formed, owing to the extremely high surface free energy of the nanoparticulate system and the thermodynamic driving force to achieve an overall reduction in free energy. With the formation of such agglomerated particles, the bioavailability of the dosage form can fall well below that observed with the liquid dispersion form of the nanoparticulate active agent.

In one embodiment, an active agent composition, e.g., is one in which administration of the composition to a subject in a fasted state is bioequivalent to administration of the composition to a subject in a non-fasted state. The difference in Cₘₐₓ and AUC_{0-∞} for the active agent composition, when administered in the non-fasted versus the fasted state, is less than about 35%, less than about 25%, less than about 20%, less than about 15%, less than about 10%, less than about 5%, or less than about 3%.

The concentration of the active agent in the oral composition can be about 99.5% to about 0.001%, about 95% to about 0.1%, or about 90% to about 0.5%, by weight, based on the total combined weight of the active agent and at least one particle sequestrant, not including other excipients. The concentration of the at least one particle sequestrant can be about 0.5% to about 99.999%, about 5.0% to about 99.9%, or about 10% to about 99.5%, by weight, based on the total combined dry weight of the active agent and at least one particle sequestrant, not including other excipients.

In another embodiment, as described above, the composition comprising active agent particles comprises a release-retarding material. Release-retarding materials can be hydrophilic and/or hydrophobic polymers. Release-retarding materials include, for example acrylic polymers, alkylcelluloses, shellac, zein, hydrogenated vegetable oil, hydrogenated castor oil, and combinations comprising one or more of the foregoing materials. The oral dosage form can contain about 1 wt % to about 80 wt % of the release-retarding material based on the total weight of the oral dosage form. Exemplary acrylic polymers include acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid-alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, and combinations comprising one or more of the foregoing polymers. The acrylic polymer can be a methacrylate copolymer with a low content of quaternary ammonium groups.

Exemplary alkylcelluloses include ethylcellulose. Those skilled in the art will appreciate that other cellulosic polymers, including other alkyl cellulosic polymers, can be substituted for part or all of the ethylcellulose.

Other exemplary hydrophobic materials are water-insoluble with more or less pronounced hydrophobic trends. The hydrophobic material can have a melting point of about 30° C. to about 200° C., more preferably about 45° C. to about 90° C. The hydrophobic material can include neutral or synthetic waxes, fatty alcohols (such as lauryl, myristyl, stearyl, cetyl or preferably cetostearyl alcohol), fatty acids, including fatty acid esters, fatty acid glycerides (mono-, di-, and tri-glycerides), hydrogenated fats, hydrocarbons, normal waxes, stearic acid, stearyl alcohol, hydrophobic and hydrophilic materials having hydrocarbon backbones, and combinations comprising one or more of the foregoing materials. Exemplary waxes include beeswax, glycowax, castor wax, carnauba wax and wax-like substances, e.g., material normally solid at room temperature and having a melting point of from about 30° C. to about 100° C., and combinations comprising one or more of the foregoing waxes.

In other embodiments, the release-retarding material can comprise digestible, long chain (e.g., C₈-C₅₀, preferably C₁₂-C₄₀), substituted or unsubstituted hydrocarbons, such as fatty acids, fatty alcohols, glyceryl esters of fatty acids, mineral and vegetable oils, waxes, and combinations comprising one or more of the foregoing materials. Hydrocarbons having a melting point of between about 25° C. and about 90° C. can be used. Of these long chain hydrocarbon materials, fatty (aliphatic) alcohols are preferred. The oral dosage form can contain up to about 60 wt % of at least one digestible, long chain hydrocarbon, based on the total weight of the oral dosage form.

Further, the sustained-release matrix or delayed release matrix can contain up to 60 wt % of at least one polyalkylene glycol.

Alternatively, the release-retarding material can comprise polylactic acid, polyglycolic acid, or a co-polymer of lactic and glycolic acid.

In another embodiment, a method of producing a solid dosage form comprises:

(a) selecting a ternary amine polymer;

(b) selecting an acidifying agent, which has a rate of dissolution suitable to facilitate dissolution of the ternary amine polymer under aqueous acidic conditions;

(c) producing particles comprising a water-insoluble active agent or an active agent with a significant food effect, and the ternary amine polymer; and

(d) combining the particles with the acidifying agent to produce the dosage form, wherein upon ingestion of the dosage form by a human subject, the acidifying agent facilitates the dissolution of the ternary amine polymer in the gastrointestinal tract.

The method is based at least in part on the following equation:

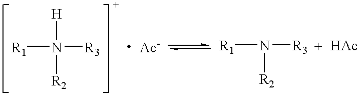

Solid dosage forms for oral administration include, but are not limited to, capsules, tablets, pills, powders, and granules. In such solid dosage forms, the active agent can be admixed with one or more of the following: (a) one or more inert excipients (or carriers), such as sodium citrate or dicalcium phosphate; (b) fillers or extenders, such as starches, lactose, sucrose, glucose, mannitol, and silicic acid; (c) binders, such as carboxymethylcellulose, alignates, gelatin, polyvinylpyrrolidone, sucrose, and acacia; (d) humectants, such as glycerol; (e) disintegrating agents, such as agar-agar, calcium carbonate, potato or tapioca starch, alginic acid, certain complex silicates, and sodium carbonate; (f) solution retarders, such as paraffin; (g) absorption accelerators, such as quaternary ammonium compounds; (h) wetting agents, such as cetyl alcohol and glycerol monostearate; (i) adsorbents, such as kaolin and bentonite; and (j) lubricants, such as talc, calcium stearate, magnesium stearate, solid polyethylene glycols, sodium lauryl sulfate, and combinations comprising one or more of the foregoing additives. For capsules, tablets, and pills, the dosage forms can also comprise buffering agents.

A method of improving the bioavailability of an active agent, comprises administering an active agent dosage form, the active agent dosage form comprising active agent nanoparticles having an average or effective average particle size of less than 2000 nm, wherein the active agent nanoparticles and a particle sequestrant are disposed on an inert core particle, and wherein the particle sequestrant is a pH-sensitive copolymer having both hydrophobic (meth)acrylate units and acid-soluble (meth)acrylate units. In one embodiment, the active agent dosage form redisperses in a biorelevant medium. In another embodiment, the active agent dosage form comprises no added surfactants or phospholipids. In yet another embodiment, the active agent dosage form comprises no added surfactant or phospholipid and redisperses in a biorelevant medium.

Benefits of an oral dosage form which substantially eliminates the effect of food include an increase in subject convenience, thereby increasing subject compliance, as the subject does not need to ensure that they are taking a dose either with or without food. This benefit is significant, as with poor subject compliance an increase in the medical condition for which the drug is being prescribed can be observed.

The invention is further illustrated by the following non-limiting example.

### Example 1

### Tablet Formulation with Acidifying Agent

A fenofibrate suspension was made according to Table 1. The fenofibrate, Eudragit® EPO and buffer were suspended in an aqueous solution comprising alcohol as a wetting agent. The Suspension was milled in a Dyno-Mill to produce the nanoparticulate suspension.

TABLE 1

| Milling suspension compositions | % Weight |
|---|---|
| Fenofibrate | 76 |
| Sodium Phosphate Monobasic Monohydrate | 12 |
| EUDRAGIT ® EPO | 12 |
| Total | 100 |

A nanoparticle suspension was made essentially as in Table 1, but with the amounts from Table 2. A fenofibrate formulation comprising 58.64 wt % of the nanoparticle suspension, salt (sodium chloride), acidifying agent (fumaric acid and succinic acid) and lubricant (magnesium stearate) were dry blended and then compressed into tablets.

The composition is given in Table 2:

TABLE 2

| Component | Weight (mg/tablet) | Wt % |
|---|---|---|
| Fenofibrate | 145.0 | 7.59 |
| Eudragit ® EPO | 220.0 | 11.52 |
| Sodium phosphate monobasic | 120.0 | 6.28 |
| ATAB, dicalcium phosphate | 1000.0 | 52.36 |
| Sodium chloride | 170.0 | 8.90 |
| Fumaric acid | 170.0 | 8.90 |
| Succinic acid | 65.0 | 3.40 |
| Magnesium stearate | 20.0 | 1.05 |
| Total | 1910.0 | 100.0 |

An “active agent” means a compound, element, or mixture that when administered to a patient, alone or in combination with another compound, element, or mixture, confers, directly or indirectly, a physiological effect on the patient. The indirect physiological effect can occur via a metabolite or other indirect mechanism. When the active agent is a compound, then salts, solvates (including hydrates) of the free compound or salt, crystalline forms, non-crystalline forms, and any polymorphs of the compound are contemplated herein. Compounds can contain one or more asymmetric elements such as stereogenic centers, stereogenic axes and the like, e.g., asymmetric carbon atoms, so that the compounds can exist in different stereoisomeric forms. These compounds can be, for example, racemates or optically active forms. For compounds with two or more asymmetric elements, these compounds can additionally be mixtures of diastereomers. For compounds having asymmetric centers, all optical isomers in pure form and mixtures thereof are encompassed. In addition, compounds with carbon-carbon double bonds can occur in Z- and E-forms, with all isomeric forms of the compounds. In these situations, the single enantiomers, i.e., optically active forms can be obtained by asymmetric synthesis, synthesis from optically pure precursors, or by resolution of the racemates. Resolution of the racemates can also be accomplished, for example, by conventional methods such as crystallization in the presence of a resolving agent, or chromatography, using, for example a chiral HPLC column. All forms are contemplated herein regardless of the methods used to obtain them.

In one embodiment, the active agent is a water insoluble active agent, such as, for example, aceclofenac, acetaminophen, acetazolamide, acetylsalicylic acid, acyclovir, albendazole, allopurinol, amoxicillin, atorvastatin, azathioprine, azithromycin, benidipine, bicalutamide, bisacodyl, cabergoline, candesartan cilexetil, carbamazepine, carvedilol, cefdinir, cefditoren pivoxil, cefixime, cefotiam hexetil hcl, cefpodoxime proxetil, cefuroxime axetil, celecoxib, chloroquine, chlorpromazine, cilostazol, clarithromycin, clofazimine, clopidogrel, clozapine, cyclosporine, cyproterone, dapsone, dexamethasone, diazepam, diclofenac, diloxanide, doxycycline, ebastine, efavirenz, epalrestat, eprosartan, erythromycin ethylsuccinate, ethylicosapentate, ezetimibe, famotidine, fenofibrate, flurbiprofen, folic acid, furosemide, gefitinib, gliclazide, glimepiride, glipizide, glyburide, griseofulvin, haloperidol, hydrochlorothiazide, hydroxyzine, ibuprofen, imatinib, indinavir, iopanoic acid, irbesartan, isotretinoin, itraconazole, ivermectin, ketoprofen, lamotrigine, l-carbocysteine, levodopa, levosulpride, linezolid, loratadine, lorazepam, lovastatin, manidipine, mebendazole, medroxyprogesteron, meloxicam, menatetrenone, mesalamine, metaxalone, methylphenidate, metoclopramide, metronidazole, modafinil, mosapride, mycophenolate mofetil, nabum etone, nalidixic acid, nelfinavir, nevirapine, nicergoline, nifedipine, nilvadipine, nimesulide, nitrofurantoin, nystatin, olanzapine, orlistat, oxcarbazepine, oxycodone, phenobarbital, phenytoin, pioglitazone, pranlukast, praziquantel, propylthiouracil, pyrantel, pyrimethamine, quetiapine, raloxifene, retinol, rifampicin, risperidone, ritonavir, roxithromycin, sennoside a, simvastatin, spironolactone, sulfadiazine, sulfamethoxazole, sulfasalazine, sultamicillin, tacrolimus, tamoxifen, telmisartan, teprenone, theophylline, ticlopidine, tocopherol nicotinate, tosufloxacin, triflusal, trimethoprim, ursodeoxycholic acid, valproic acid, valsartan, verapamil, warfarin, or zaltoprofen.

In another embodiment, the active agent is an active agent that exhibits a food effect such as, for example, estradiol, norethindrone acetate, lovastatin, niacin, aspirin, dipyridamole, diclofenac sodium, misoprostol, amoxicillin, clavulanate potassium, atovaquone, proguanil hydrochloride, hydrochlorothiazide, telmisartan, carbidopa, levodopa, estrogens, conjugated, medroxyprogesterone acetate, lansoprazole, naproxen, amoxicillin, clarithromycin, lansoprazole, interferon alfa-2b, recombinant, ribavirin, trandolapril, verapamil hydrochloride, aripiprazole, zafirlukast, isotretinoin, amprenavir, fexofenadine hydrochloride, zolpidem tartrate, lubiprostone, cyclobenzaprine hydrochloride, tipranavir, rasagiline mesylate, acamprosate calcium, carbamazepine, cefuroxime axetil, celecoxib, tolterodine tartrate, valsartan, isradipine, selegiline, hydrochloride, raloxifene hydrochloride, rivastigmine tartrate, deferasirox, tamsulosin hydrochloride, metformin hydrochloride, alendronate sodium, cyclosporine, ziprasidone mesylate, ziprasidone hydrochloride, sumatriptan succinate, etravirine, digoxin, mefloquine hydrochloride, vardenafil hydrochloride, levothyroxine sodium, atorvastatin calcium, atovaquone, methylphenidate hydrochloride, mycophenolic acid, cyclosporine, gabapentin, esomeprazole magnesium, oxymorphone hydrochloride, oxymorphone hydrochloride, doxycycline monohydrate, cilostazol, tacrolimus, progesterone, sirolimus, atazanavir sulfate, propafenone hydrochloride, cinacalcet hydrochloride, quetiapine fumarate, metaxalone, ivermectin, levothyroxine sodium, erlotinib, tolcapone, ticlopidine hydrochloride, theophylline anhydrous, valganciclovir hydrochloride, nelfinavir mesylate, tenofovir disoproxil fumarate, or sodium oxybate.

By “substantially water-insoluble” or “poorly soluble” active agent, it is meant an agent having a water solubility of less than 1 mg/ml.

“Efficacy” means the ability of an active agent administered to a patient to produce a therapeutic effect in the patient.

“Safety” means the incidence or severity of adverse events associated with administration of an active agent, including adverse effects associated with patient-related factors (e.g., age, gender, ethnicity, race, target illness, abnormalities of renal or hepatic function, co-morbid illnesses, genetic characteristics such as metabolic status, or environment) and active agent-related factors (e.g., dose, plasma level, duration of exposure, or concomitant medication).

A “dosage form” means a unit of administration of an active agent. Examples of dosage forms include tablets, capsules, injections, suspensions, liquids, emulsions, creams, ointments, suppositories, inhalable forms, transdermal forms, and the like. A “treatment form” refers to a dosage form that is bioequivalent to a current commercially available oral formulation.

“Bioavailability” means the extent or rate at which an active agent is absorbed into a living system or is made available at the site of physiological activity. For active agents that are intended to be absorbed into the bloodstream, bioavailability data for a given formulation can provide an estimate of the relative fraction of the administered dose that is absorbed into the systemic circulation. “Bioavailability” can be characterized by one or more pharmacokinetic parameters.

“Pharmacokinetic parameters” describe the in vivo characteristics of an active agent (or surrogate marker for the active agent) over time, such as plasma concentration (C), Cₘₐₓ, Cₙ, C₂₄, Tₘₐₓ, and AUC. “Cₘₐₓ” is the measured concentration of the active agent in the plasma at the point of maximum concentration. “Cₙ” is the measured concentration of an active agent in the plasma at about n hours after administration. “C₂₄” is the measured concentration of an active agent in the plasma at about 24 hours after administration. The term “Tₘₐₓ” refers to the time at which the measured concentration of an active agent in the plasma is the highest after administration of the active agent. “AUC” is the area under the curve of a graph of the measured concentration of an active agent (typically plasma concentration) vs. time, measured from one time point to another time point. For example AUC₀₋ₜ is the area under the curve of plasma concentration versus time from time 0 to time t. The AUC_{0-∞} or AUC_{0-INF} is the calculated area under the curve of plasma concentration versus time from time 0 to time infinity.

Food is typically a solid food with sufficient bulk and fat content that it is not rapidly dissolved and absorbed in the stomach. In one embodiment, “food” is a meal, such as breakfast, lunch, or dinner. The terms “taken with food,” “fed” and “non-fasted” are equivalent and are as given by FDA guidelines and criteria. In one embodiment, “with food” means that the dosage form is administered to a patient between about 30 minutes prior to about 2 hours after eating a meal. In another embodiment, “with food” means that the dosage is administered at substantially the same time as the eating the meal.

The terms “without food,” “fasted,” and “an empty stomach” are equivalent and are as given by FDA guidelines and criteria. In one embodiment, “fasted” means the condition of not having consumed solid food for at least about 1 hour prior or at least about 2 hours after such consumption. In another embodiment, “fasted” means the condition of not having consumed solid food for at least about 1 hour prior to at least about 2 hours after such consumption.

For the purposes of biostudy and the determination of bioequivalence, a “fasted patient” means a patient who does not eat any food, i.e., fasts, for at least 10 hours before the administration of a dosage form of active agent and who does not eat any food and continues to fast for at least 4 hours after the administration of the dosage form. The dosage form is administered with 240 ml of water during the fasting period, and water can be allowed ad libitum after 2 hours.

For the purposes of biostudy and the determination of bioequivalence, a “non-fasted patient” means a patient who fasts for at least 10 hours overnight and then consumes an entire test meal within 30 minutes of first ingestion. The dosage form is administered with 240 mL of water at 30 minutes after first ingestion of the meal. No food is then allowed for at least 4 hours post-dose. Water can be allowed ad libitum after 2 hours. A high fat test meal provides approximately 1000 calories to the patient of which approximately 50% of the caloric content is derived from fat content of the meal. A representative high fat high calorie test meal comprises 2 eggs fried in butter, 2 strips of bacon, 2 slices of toast with butter, 4 ounces of hash brown potatoes, and 8 ounces of whole milk to provide 150 protein calories, 250 carbohydrate calories, and 500 to 600 fat calories.

Under U.S. FDA guidelines, two products or methods (e.g., dosing under non-fasted versus fasted conditions) are bioequivalent if the 90% Confidence Intervals (CI) for the ratios of a log transformed geometric mean of AUC_{0-∞} for the first product or method compared to the second product or method, and Cₘₐₓ for the first product or method compared to the second product or method, are within 0.80 to 1.25 (Tₘₐₓ measurements are not relevant to bioequivalence for regulatory purposes). To show bioequivalency between two compositions or methods pursuant to Europe's EMEA guidelines, the 90% CI for the ratios of a log transformed geometric mean of AUC_{0-∞} for the first product or method compared to the second, must be within 0.80 to 1.25 and the 90% CI for the ratios of a log transformed geometric mean of Cₘₐₓ for the first product or method compared to the second must be within 0.70 to 1.43

The terms “a” and “an” do not denote a limitation of quantity, but rather denote the presence of at least one of the referenced item. The term “or” means “and/or.” The terms “comprising,” “having,” “including,” and “containing” are to be construed as open-ended terms (i.e., meaning “including, but not limited to”). Unless defined otherwise, technical and scientific terms used herein have the same meaning as is commonly understood by one of skill in the art to which this invention belongs. The endpoints of all ranges directed to the same component or property are inclusive and independently combinable.

Embodiments of this invention are described herein, including the best mode known to the inventors for carrying out the invention. Variations of those preferred embodiments would become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An active agent composition comprising
particles comprising a water-insoluble active agent or an active agent with a significant food effect; and a ternary amine polymer having both hydrophobic (meth)acrylate units and acid-soluble (meth)acrylate units, and
an acidifying agent;
wherein upon ingestion by a human subject, the acidifying agent facilitates the dissolution of the ternary amine polymer in the gastrointestinal tract.

2. The active agent composition of claim 1, wherein the particles are suspended in a matrix comprising the acidifying agent.

3. The active agent composition of claim 1, wherein particles comprise nanoparticles or microparticles disposed on an inert core.

4. The active agent composition of claim 3, wherein the inert core particles have an average diameter of 5 to 500 μm.

5. The active agent composition of claim 1, wherein the a ternary amine polymer comprises a butyl methacrylate-(2-dimethylaminoethyl)methacrylate-methyl methacrylate copolymer.

6. The active agent composition of claim 1, further comprising an ionic strength modifying agent.

7. The active agent composition of claim 1, wherein the acidifying agent is an organic acid

8. The active agent composition of claim 7, wherein the organic acid is citric acid, maleic acid, fumaric acid, succinic acid, or a combination thereof.

9. The active agent composition of claim 7, wherein the organic acid is fumaric acid and succinic acid.

10. The active agent composition of claim 7, wherein the ratio of the organic acid to the ternary amine polymer is about 0.1 to about 10.0, specifically about 0.3 to about 2.

11. The active agent composition of claim 10, wherein the ratio of the organic acid to the ternary amine polymer is about 0.3 to about 2.

12. A method of producing a solid dosage form, comprising:
(a) selecting a ternary amine polymer;
(b) selecting an acidifying agent which has a rate of dissolution suitable to facilitate dissolution of the ternary amine polymer under aqueous acidic conditions;
(c) producing particles comprising a water-insoluble active agent or an active agent with a significant food effect, and the ternary amine polymer; and
(d) combining the particles with the acidifying agent to produce the dosage form, wherein upon ingestion of the dosage form by a human subject, the acidifying agent facilitates the dissolution of the ternary amine polymer in the gastrointestinal tract.
